(19) United States
(12) Patent Application Publication
(10) Pub. No.: US 2025/0250452 A1
(43) Pub. Date: Aug. 7, 2025

(54) DISINFECTANT VISUALIZING INK AND DISINFECTANT VISUALIZING SHEET

(71) Applicant: CANON KABUSHIKI KAISHA, Tokyo (JP)

(72) Inventors: KOROMO SHIROTA, Kanagawa (JP); MASANORI SEKI, Kanagawa (JP); HIROSHI SAITO, Kanagawa (JP); MASARU SUGITA, Tokyo (JP)

(21) Appl. No.: 19/188,796

(22) Filed: Apr. 24, 2025

Related U.S. Application Data
(63) Continuation of application No. PCT/JP2023/036463, filed on Oct. 6, 2023.

(30) Foreign Application Priority Data
Oct. 26, 2022 (JP) 2022-171842

(51) Int. Cl. C09D 11/50; A61L 2/28; C09D 11/037; C09D 11/328; G01N 33/52

(52) U.S. Cl. CPC C09D 11/50; A61L 2/28; C09D 11/037; C09D 11/328; G01N 33/528

(57) ABSTRACT

A disinfectant visualizing ink includes a visualizing agent that changes in color state when a disinfectant is applied and is hardly soluble in an aqueous liquid, a dispersant that disperses the visualizing agent in the aqueous liquid, and the aqueous liquid.

### CROSS-REFERENCE TO RELATED APPLICATIONS

This application is a Continuation of International Patent Application No. PCT/JP2023/036463, filed Oct. 6, 2023, which claims the benefit of Japanese Patent Application No. 2022-171842, filed Oct. 26, 2022, both of which are hereby incorporated by reference herein in their entirety.

### BACKGROUND OF THE INVENTION

### Field of the Invention

The present invention relates to a disinfectant visualizing ink and a disinfectant visualizing sheet.

### Background Art

In recent years, the risk of infectious diseases has increased, and cities have been locked down in consideration of the medical system. In daily life, there are more and more cases where face-to-face conversations are not possible, and in highly public spaces such as coworking spaces, the occupancy rate has decreased, the productivity of users of those facilities has decreased, and business opportunities have been lost. There are concerns that this will ultimately lead to a decline in the economy.

Therefore, the act of cleaning and sterilization has been considered important, and technologies for visualizing the act have been disclosed. Patent Document 1 discusses a technique for visualizing a sterilizing operation during cleaning using a fluorescent material in the ultraviolet region. However, this technology requires special tools and is thus not convenient.

Patent Document 2 discusses a technique for changing the color of a pH-responsive dye by reaction with a disinfectant. However, this method requires mixing two reactive substances into one liquid, making it difficult to store the product for a long period of time.

Patent Documents 1 and 2 discuss materials that can visualize the application of a disinfectant, but they do not discuss how to record or manage the application status of the disinfectant when there are many sterilization targets. When there are many sterilization targets, if a person records the application of the disinfectant by handwriting or by inputting into a computer, it is time-consuming and there is a possibility that the application status may be recorded incorrectly.

Patent Document 3 discusses a technique for coloring a material by embedding porous detection particles in the surface of the material. However, since the detection species is not water-soluble, a paste-like material such as ultraviolet ink is deposited in advance after the detection species is sprayed. Accordingly, the resolution is insufficient.

### CITATION LIST

### Patent Literature

PTL 1: U.S. Pat. No. 8,519,360
PTL 2: United States Patent Application Publication No. 2017-0336373
PTL 3: Japanese Patent Laid-Open No. 2020-101535

### SUMMARY OF THE INVENTION

In view of this, the present invention is directed to providing ink that allows visual confirmation that a disinfectant has been applied.

According to an aspect of the present invention, a disinfectant visualizing ink includes a visualizing agent that changes in color state when a disinfectant is applied and is hardly soluble in an aqueous liquid, a dispersant that disperses the visualizing agent in the aqueous liquid, and the aqueous liquid.

Further features of the present invention will become apparent from the following description of exemplary embodiments with reference to the attached drawings.

### BRIEF DESCRIPTION OF THE DRAWINGS

FIG. 1 is a schematic diagram of a disinfectant visualizing sheet according to a first exemplary embodiment of the present invention.

FIG. 2 is a schematic diagram illustrating an example of a disinfectant visualizing sheet according to the first exemplary embodiment of the present invention.

### DESCRIPTION OF THE EMBODIMENTS

Hereinafter, an exemplary embodiment of the present invention will be described. However, prior to describing an exemplary embodiment of an aqueous ink for manufacturing a disinfectant visualizing sheet, an exemplary embodiment of a disinfectant visualizing sheet that uses a disinfectant visualizing ink according to the present exemplary embodiment will be described.

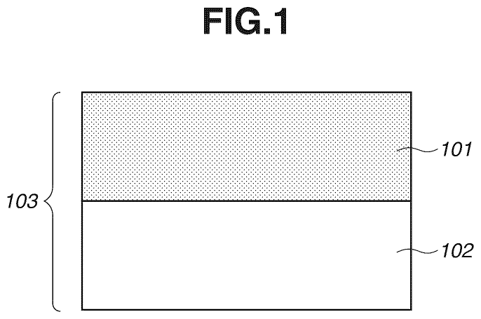

In the present exemplary embodiment, a disinfectant visualizing sheet 103 has a base material 102 and a chromogenic portion 101 provided on the base material 102 (FIG. 1). The chromogenic portion 101 has a visualizing agent (not illustrated) that changes in color state when a disinfectant is applied. Using the disinfectant visualizing sheet according to the present exemplary embodiment makes it possible to conveniently confirm the application of the disinfectant by visually checking the change in the color state. In addition, the disinfectant and the visualizing agent are mixed only when the disinfectant is applied to the disinfectant visualizing sheet, and the time during which the disinfectant and the visualizing agent are mixed is short. Therefore, the materials used can be easily stored for a long period of time.

The application of the disinfectant here means that the disinfectant reaches the visualizing agent, and includes spraying the disinfectant and wiping with a duster containing the disinfectant. In the disinfection process by wiping with a duster, sodium hypochlorite, ethanol water, and the like are widely used as the disinfectant.

The disinfectant visualizing ink according to the present exemplary embodiment includes a visualizing agent that changes in color state when the disinfectant is applied and is hardly soluble in an aqueous liquid, a dispersant that disperses the visualizing agent in the aqueous liquid, and the aqueous liquid.

In the present exemplary embodiment, the dispersant preferably has at least one functional group selected from the group consisting of a polyethylene oxide group, a polypropylene oxide group, and a polyglycerin group. The dispersant also preferably has a copolymer containing a repeating unit derived from ethylene oxide and a repeating unit derived from propylene oxide.

In the present exemplary embodiment, when the total mass of the visualizing agent contained in the ink is taken as 100% by mass, the content of the dispersant in the ink is preferably 30% by mass or more and 200% by mass or less.

In the present exemplary embodiment, the visualizing agent preferably contains at least one selected from the group consisting of orthocresolphthalein, thymol blue, and thymolphthalein. When the total mass of the ink is taken as 100% by mass, the content of the visualizing agent in the ink is preferably 1% by mass or more and 10% by mass or less.

The disinfectant visualizing ink of the present exemplary embodiment is an ink for forming a chromogenic portion in the disinfectant visualizing sheet, and can be applied to the base material by various methods such as spraying and coating. Among them, forming the chromogenic portion by the inkjet method makes it possible to display a character barcode or the like on demand with high resolution on the chromogenic portion.

The details of each part of the disinfectant visualizing sheet will be described below.

Hereinafter, the aqueous ink for manufacturing the disinfectant visualizing sheet of the present exemplary embodiment will be described.

### <Visualizing Agent>

The visualizing agent in the present exemplary embodiment is not particularly limited as long as it is hardly soluble in water and changes in color state by reaction with a disinfectant described below. In the present exemplary embodiment, the change in color state refers to a change by which the difference in color between before and after the application of the disinfectant can be visually confirmed. For example, the change in color state can be a change from a state of being invisible to the naked eye to a state of being visible to the naked eye, a change from a state of being visible to the naked eye to a state of being invisible to the naked eye, or a color change that is visually recognizable. Examples of the visualizing agent in the present exemplary embodiment include o-cresolphthalein and thymolphthalein.

Visually recognizable color changes here include changes in color shades and changes in hue. A change in color shade is a change from dark red to light red, for example, and a change in hue is a change from red to blue, for example. Visually recognizable color changes also include, for example, a change from colorless to colored colors and vice versa.

The visualizing agent may be a combination of a plurality of materials that exhibit different changes in color state.

A phenomenon of changing the color state includes chromism. Examples of chromism include photochromism, thermochromism, electrochromism, acidichromism, solvatochromism, and vapochromism. In the present exemplary embodiment, substances that cause these phenomena are called chromic substances, and can be specifically called photochromic substances, thermochromic substances, electrochromic substances, acidichromic substances, solvatochromic substances, or vapochromic substances.

The visualizing agent of the present exemplary embodiment is one that is hardly soluble in water among the above-mentioned substances. In this specification, hardly soluble is defined as having a solubility of 0.1% or less in pure water at 23° C.

The content of the visualizing agent is preferably 1 to 10% with respect to the total mass of the ink composition in order to obtain a deep color.

### <Dispersant>

The disinfectant visualizing ink in the present exemplary embodiment contains a dispersant for dispersing the visualizing agent in water. If the visualizing agent develops a color by acidichromism, the dispersant preferably has at least one functional group selected from the group consisting of a polyethylene oxide group, a polypropylene oxide group, and a polyglycerin group. This is because acidichromism requires the mediation of water, and it is presumed that the presence of these functional groups in the vicinity of the visualizing agent makes it easier for water to be attracted, which facilitates color development. In addition, the water-retention effect of these functional groups makes it easier for the visualizing agent to continue color development. In particular, if the visualizing agent is thymol blue or orthocresolphthalein (o-cresolphthalein), which develops color by acidichromism, a dispersant having one or more functional groups selected from a polyethylene oxide group, a polypropylene oxide group, and a polyglycerin group is suitable for the above reasons.

On the other hand, if a highly hydrophobic dispersant is used, such as a styrene-maleic acid copolymer with a molecular weight of 10,000 or more, the dispersant may cover the visualizing agent in the color development area, inhibiting color development by the disinfectant.

Preferred as the dispersant having one or more functional groups selected from a polyethylene oxide group, a polypropylene oxide group, and a polyglycerin group, are EO-PO block polymer-based dispersants mainly constituted of a copolymer of ethylene oxide (EO) and propylene oxide (PO) (trade names: DisperBYK 183, DisperBYK185, DisperBYK190, and the like, manufactured by BYK-Chemie GmbH), decaglycerin-based dispersants (trade name: NIKKOL Decaglyn 1-ISV and the like, manufactured by Nikko Chemicals Co., Ltd.), an ethylene oxide-based dispersant (trade name: EMULGEN 420 manufactured by Kao Corporation), and the like. Among these, EO-PO block polymer-based dispersants are preferred.

These dispersants are used in an amount of 30% by mass to 200% by mass when the total mass of the visualizing agent is taken as 100% by mass, and are dispersed by a conventionally known method to form ink components.

### <Aqueous Liquid>

The aqueous ink for manufacturing a disinfectant visualizing sheet of the present invention is an aqueous ink containing at least water as an aqueous liquid. The ink can contain water or an aqueous liquid that is a mixed solvent of water and a water-soluble organic solvent. The water is preferably deionized water or ion-exchanged water.

The content (% by mass) of water in the ink is preferably 50.00% by mass or more and 95.00% by mass or less with respect to the total mass of the ink. The content (% by mass) of the water-soluble organic solvent in the ink is preferably 3.00% by mass or more and 50.00% by mass or less with respect to the total mass of the ink. The water-soluble organic solvent may be any of water-soluble organic solvents usable for inkjet ink, such as alcohols, (poly)alkylene glycols, glycol ethers, nitrogen-containing compounds, and sulfur-containing compounds.

### (Other Additives)

In addition to the above-described components, the aqueous ink for manufacturing a disinfectant visualizing sheet of the present invention may contain water-soluble organic compounds including polyhydric alcohols such as trimethylolpropane and trimethylolethane, and urea, and urea derivatives such ethyleneurea, as necessary. The marking composition in the present exemplary embodiment may contain various additives such as surfactants, pH adjusters, rust inhibitors, preservatives, antifungal agents, antioxidants, reduction inhibitors, evaporation promoters, chelating agents, and water-soluble resins, as necessary.

In the case of discharging the aqueous ink for manufacturing a disinfectant visualizing sheet of the present invention from an inkjet recording head to record an image on a recording medium, it is preferable to use the ink of which the surface tension and viscosity are appropriately controlled. Specifically, the surface tension of the composition at 25° C. is preferably 10 mN/m or more and 60 mN/m or less, more preferably 20 mN/m or more and 60 mN/m or less, and particularly preferably 30 mN/m or more and 50 mN/m or less.

The viscosity of the composition at 25° C. is preferably 1.0 mPa's or more and 10 mPa's or less, and more preferably 1.0 mPa's or more and 5 mPa's or less.

### <Image Recording Method (Method of Applying Aqueous Ink for Manufacturing Disinfectant Visualizing Sheet)>

An image recording method in the present exemplary embodiment may be a spray method, an inkjet method, or the like.

For example, the inkjet method is a method by which the ink of the present invention is discharged from an inkjet recording head to record an image on a recording medium. Methods for discharging the ink include a method of applying mechanical energy and a method of applying thermal energy to the composition. Any known method can be used for the steps of the inkjet recording method except for using the ink of the present invention.

Two or more types of visualizing agents may be used. For example, it is desirable to use visualizing agents that maintain color development for different periods of time, since this can serve as an indicator to show the time since the disinfectant came into contact.

The visualizing agent may be provided to show text information, image information (figure), or both image information and text information. Although the text information and image information are not particularly limited, it is considered that if the text information and image information are displayed in a way that satisfies the user of the sterilized area, the user will be more likely to apply the disinfectant voluntarily. The visualizing agent changes color and recovers as the disinfectant evaporates. The recovery here means returning to the state before application of the disinfectant. That is, the color state is reversible. That is, the visualizing agent in the present exemplary embodiment can change in color state for a certain period of time after application of the disinfectant, and then return to the state before application of the disinfectant. For example, the color state of the visualizing agent invisible to the naked eye can be changed to be visible to the naked eye by application of the disinfectant, and after a certain period of time, the color state can be returned to be invisible to the naked eye. Accordingly, the visualizing agent is reversible, and can be used in situations where repeated sterilization is required.

### (Disinfectant Visualizing Sheet)

The disinfectant visualizing sheet 103 according to the present exemplary embodiment has the base material 102 and the chromogenic portion 101 provided on the base material 102 (FIG. 1). The chromogenic portion 101 has a visualizing agent (not illustrated) that changes in color state when a disinfectant is applied. Using the disinfectant visualizing sheet 103 according to the present exemplary embodiment makes it possible to conveniently confirm the application of the disinfectant by visually checking the change in the color state. In addition, the disinfectant and the visualizing agent are mixed only when the disinfectant is applied to the disinfectant visualizing sheet 103, and the time during which the disinfectant and the visualizing agent are mixed is short. Therefore, the materials used can be easily stored for a long period of time. The application of the disinfectant here means that the disinfectant reaches the visualizing agent, and includes spraying the disinfectant and wiping with a duster containing the disinfectant.

### <Chromogenic Portion 101>

The chromogenic portion 101 in the present exemplary embodiment contains at least a visualizing agent. The chromogenic portion 101 may contain a binder to improve adhesion to the base material 102. The binder may be a resin, and may be a urethane resin or polyvinyl alcohol, for example.

In the present exemplary embodiment, the thickness of the chromogenic portion 101 is preferably 1 μm or more and 80 μm or less, more preferably 5 μm or more and 60 μm or less, and further more preferably 15 μm or more and 50 μm or less. Setting the thickness of the chromogenic portion 101 to 80 μm or less improves the adhesion to the base material 102, and setting the thickness of the chromogenic portion 101 to 1 μm or more improves the color state of the chromogenic portion 101.

The chromogenic portion 101 in the present exemplary embodiment may be provided on at least a portion of the base material 102, or may be provided on the entire surface of the base material 102. Examples of the chromogenic portion 101 provided on at least a portion of the base material 102 include a polka dot pattern or a checkerboard pattern. The pattern of the chromogenic portion 101 may indicate the starting position and ending position of wiping with a disinfectant. In a case where the chromogenic portion 101 is to be provided on a rectangular base material 102, the pattern may be formed such that the area of the chromogenic portion 101 near the center is smaller than that of the four corners.

### <Base Material 102>

The base material 102 in the present exemplary embodiment may be any base material 102 on which a chromogenic portion 101 can be provided.

The base material 102 is preferably a resin sheet because it is lightweight and flexible. Examples of the material for the resin sheet include polyester resins such as polyethylene terephthalate, polybutylene terephthalate, and polyethylene terephthalate/isophthalate copolymers; polyolefin resins such as polyethylene, polypropylene, and polymethylpentene; polyethylene fluoride resins such as polyvinyl fluoride, polyvinylidene fluoride, polyethylene tetrafluoride, and ethylene-tetrafluoride ethylene copolymers; aliphatic polyamide resins such as nylon 6 and nylon 6,6; vinyl polymer resins such as polyvinyl chloride, vinyl chloride/vinyl acetate copolymers, ethylene/vinyl acetate copolymers, ethylene/vinyl alcohol copolymers, polyvinyl alcohol, and vinylon; cellulose resins such as cellulose triacetate and cellophane; acrylic resins such as polymethyl methacrylate, polyethyl methacrylate, polyethyl acrylate, and polybutyl acrylate; other synthetic resins such as polystyrene, polycarbonate, polyarylate, and polyimide, and the like. A single type of resin sheet may be used or two or more types of resin sheets may be used in a composite or laminated form of two. The base material 102 is preferably provided with a receiving layer that holds the chromogenic portion 101. This is because if the visualizing agent is ink, it is easier for the receiving layer to hold the ink.

In the present exemplary embodiment, it is preferable that the receiving layer of the base material 102 does not react with the visualizing agent. For example, if the visualizing agent develops a color in an alkaline state, the receiving layer is desirably neutral or acidic. If the receiving layer reacts with the visualizing agent, the receiving layer will undesirably develop a color before reacting with the disinfectant.

The base material 102 in the present exemplary embodiment may be transparent, opaque, or colored. If the visualizing agent has a color before it reacts with the disinfectant, the base material 102 desirably has a color close to the color of the visualizing agent.

In the present exemplary embodiment, the base material 102 may be a release paper, a metal plate, wood, or the like. The base material 102 may have an adhesive part for adhesion to another member. The adhesive part may be provided on the entire surface of the base material 102, or may be provided on a portion of the base material 102. The base material 102 with a partial adhesive part would be easy to peel off. The adhesive part may contain a UV-curable resin to facilitate peeling.

If the base material 102 is paper, the chromogenic portion 101 may contain an anionic, cationic, nonionic, or other surfactant in order to make the visualizing agent more compatible with the paper. The surfactant is preferably a nonionic surfactant such as polyoxyethylene alkyl ether, polyoxyethylene fatty acid ester, polyoxyethylene alkyl phenyl ether, polyoxyethylene-polyoxypropylene block copolymer, or acetylene glycol compound.

### <Holding Portion>

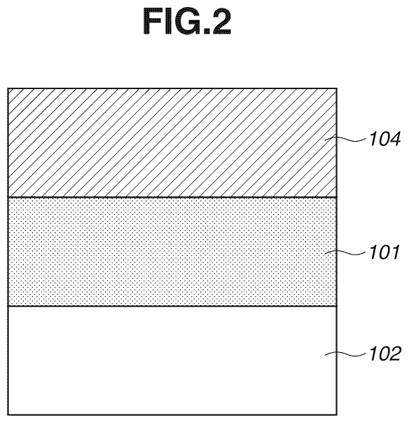

The disinfectant visualizing sheet 103 in the present exemplary embodiment may have a holding portion 104 that holds a disinfectant on the chromogenic portion 101 (FIG. 2). The holding portion 104 can hold the disinfectant for a long time, and therefore can extend the time for the disinfectant to react with the visualizing agent and develop a color. In addition, the color development time can be controlled by appropriately selecting a material for the holding portion 104. For example, the time for change from a visible color state to an invisible color state can be controlled.

Because of such a function, the holding portion 104 can also be called a decoloring time control portion.

The holding portion 104 desirably has voids to hold the applied disinfectant. For example, the holding portion 104 may include a plurality of inorganic particles, and voids may be formed between the plurality of inorganic particles. The inorganic particles used here are at least one selected from the group consisting of pearl necklace-shaped silica particles, chain-shaped silica particles, spherical colloidal silica, non-spherical colloidal silica, alumina particles, titania particles, and zirconia particles. The peak position (hereinafter, simply referred to as particle size) of particle size distribution of the inorganic fine particles obtained by a dynamic light scattering method is preferably 1 nm or more and 300 nm or less, more preferably 1 nm or more and 150 nm or less, and particularly preferably 1 nm or more and 60 nm or less. In particular, the particle size of the pearl necklace-shaped silica particles is preferably 20 nm or more and 50 nm or less. In addition, the particle size of the chain-shaped silica particles is desirably 1 nm or more and 10 nm or less. Setting the particle size to 300 nm or less sufficiently secures the amount of voids, and setting the particle size to 1 nm or more makes sufficiently large the pore diameter of the voids formed by the inorganic particles, so that the absorption speed of the applied disinfectant can be increased. The pore diameter of the voids has preferably a maximum peak in the range of 5 nm to 20 nm in a pore diameter distribution curve. If the pore diameter peak is 5 nm or more, the absorption speed of the disinfectant increases, and if the pore diameter peak exceeds 20 nm, the haze may increase. As the haze increases, the whitishness will become noticeable, so the pore diameter peak is desirably 20 nm or less in order to reduce the decrease in the visibility of the chromogenic portion 101. The pore diameter can be measured by BET specific surface area measurement.

The resin used here can be selected from among various resins. If the disinfectant is water-based, a water-soluble resin is preferred. The water-soluble resin can be at least one selected from the group consisting of cellulose-based binders such as methyl cellulose, methyl hydroxyethyl cellulose, methyl hydroxypropyl cellulose, and hydroxyethyl cellulose, natural polymer resins or derivatives thereof such as starch and its modified products, gelatin and its modified products, casein, pullulan, gum arabic, karaya gum, and albumin, polyvinyl alcohol and its modified products, latexes and emulsions such as styrene-butadiene rubber (SBR) latex, nitril-butadiene rubber (NBR) latex, methyl methacrylate-butadiene copolymer, and ethylene-vinyl acetate copolymer, vinyl polymers such as polyacrylamide and polyvinylpyrrolidone, polyethyleneimine, polypropylene glycol, polyethylene glycol, and maleic anhydride or its copolymers, for example.

The thickness of the holding portion 104 does not have to be uniform. For example, the holding time of the disinfectant can be changed by changing the thickness of a portion of the holding portion 104. This makes it possible to change the color development time of the chromogenic portion 101, and display the time from contact with the disinfectant as an indicator.

### <Protection Portion>

In the present exemplary embodiment, a protection portion may be provided on the chromogenic portion 101 to protect the chromogenic portion 101. Regarding the place on the chromogenic portion 101, the protection portion may be provided in contact with the chromogenic portion 101, or if the holding portion 104 is provided on the chromogenic portion 101, the protection portion may be provided on the holding portion 104. For example, if a water-soluble resin is used as the holding portion 104, its surface will be viscous and thus is preferably coated with a protective layer. The protection portion in the present exemplary embodiment can be made of at least one selected from the group consisting of resins such as acrylic resin, vinyl acetate resin, vinyl chloride resin, ethylene/vinyl acetate copolymer resin, polyamide resin, polyester resin, urethane resin, polyolefin resin, and copolymer resins thereof. In addition, the protection portion is preferably configured to transmit the disinfectant sufficiently to cause the visualizing agent to develop a color.

The protection portion in the present exemplary embodiment may have a water-resistant material that improves water resistance. The water-resistant material may be a binder that is highly hydrophobic and difficult to hydrolyze, with a reduced amount of a hydrophilic resin. Specific examples of the water-resistant material include an acrylic resin, polycarbonate-modified urethane resin, and polyether-modified urethane resin.

One of these may be used alone, or two or more may be used in combination.

The protection portion preferably contains the binder hard to hydrolyze such that the content of the water-soluble resin relative to the total content of the binder in the ink receiving layer is 20% by mass or less.

The protection portion in the present exemplary embodiment may have a weather-resistant material that improves weather resistance. The weather-resistant material can be any of hindered amine-based compounds such as benzophenone-based, benzotriazole-based, and triazine-based compounds, which are ultraviolet absorbents or light stabilizers.

In addition, the decolorization time control portion more preferably provides durability since the surface of the disinfectant visualizing sheet 103 is repeatedly wiped with the disinfectant.

### <Tank>

An ink tank according to the present exemplary embodiment contains the disinfectant visualizing ink according to the present exemplary embodiment.

### EXAMPLES

The present invention will be described in more detail below with reference to examples and comparative examples, but the present invention is not limited to the following examples unless it deviates from the gist of the invention. With regard to the amounts of components, “parts” and “%” are based on mass unless otherwise specified.

### Preparation of Example 1

Visualizing agent: O-cresolphthalein: 2 parts
Dispersant: DisperBKY 190 manufactured by BYK-Chemie GmbH: 0.8 parts
Ion-exchanged water: 16 parts

The above was dispersed (planetary ball mill manufactured by Fritsch GmbH, 300 rpm, four hours, medium: zirconia beads, 0.5 mmΦ, medium filling rate: 70%), diluted with a 15% aqueous glycerin solution such that the visualizing agent concentration became 5%, stirred with a stirrer for one hour, and then filtered through a 0.5 μm filter to obtain ink of Example 1.

Examples 2 to 9 were obtained in a similar manner as Example 1, except that the visualizing agent, dispersant, and visualizing agent concentration were changed as illustrated in Table 1.

In addition, ink of Comparative Example 1 was obtained in a similar manner as Example 1 except that thymolphthalein was used as a visualizing agent and no dispersant was used.

TABLE 1

| | | | | Concentration |
|---|---|---|---|---|
| | | | | of visualizing |
| | | | Dispersant/visualizing | agent in ink |
| Example | Visualizing Agent | Dispersant | agent (%) | (%) |
| Example 1 | o-Cresolphthalein | BYK-190 by BYK- | 40% | 5% |
| | | Chemie | | |
| Example 2 | o-Cresolphthalein | BYK-184 by BYK- | 50% | 6 |
| | | Chemie | | |
| Example 3 | o-Cresolphthalein | Nikkol Decaglycerin | 70% | 3 |
| | | 1-ISV by Nikko | | |
| | | Chemicals | | |
| Example 4 | o-Cresolphthalein | Nikkol Decaglycerin | 80% | 2 |
| | | 1-ISV by Nikko | | |
| | | Chemicals | | |
| Example 5 | o-Cresolphthalein | BYK-190 by BYK- | 30% | 8 |
| | | Chemie | | |
| Example 6 | o-Cresolphthalein | Emulgen 420 by | 200% | 1 |
| | | Kao | | |
| Example 7 | Thymolphthalein | BYK-190 by BYK- | 100% | 5 |
| | | Chemie | | |
| Example 8 | Thymolphthalein | Emulgen 420 by | 50% | 6 |
| | | Kao | | |
| Example 9 | o-Cresolphthalein | Joncryl HPD-671 by | 50% | 3 |
| | | BASF Japan | | |
| | | (styrene-acrylic acid | | |
| | | polymer dispersant) | | |
| | | was dissolved to | | |
| | | make a neutral | | |
| | | dispersion. | | |
| Comparative | Thymolphthalein | None | — | 0.3 |
| Example 1 | | | | |

### Preparation of Disinfectant Visualizing Sheets 1 to 8

The ink of Example 1 was charged into a yellow cartridge of an inkjet recording apparatus (EP-M553T manufactured by Epson Corporation) to create a solid image with 100% yellow duty using image processing software (Photoshop (registered trademark) manufactured by Adobe Inc.). Glossy paper (product name: Photo Paper Pro PT-201 manufactured by Canon Inc.) was used as the base material. The obtained recorded matter was dried for 24 hours in an environment with a temperature of 23° C. and a relative humidity of 55%.

This is referred to as disinfectant visualizing sheet 1.

Disinfectant visualizing sheets 2 to 9 were prepared in a similar manner as above except that the ink was changed to the ink of Example 2, and a disinfectant visualizing sheet 10 was created using the ink of Comparative Example 1.

### <Evaluation>

A 0.5 wt % aqueous solution of sodium hypochlorite was used as the disinfectant. The disinfectant was dropped 1 ml onto a Kimwipe, and the disinfectant was applied to each disinfectant visualizing sheet with the Kimwipe. The sheet was placed on white paper and filmed with a video. The filmed video was converted into a grayscale of 0 to 255, and the absolute value of the change in value between before and after the application was measured. Among the following evaluation criteria, “A” and “B” are acceptable levels, and “C” is an unacceptable level.
A: 30 or more
B: 15 or more and less than 30
C: Less than 15

TABLE 2

| | | Change N in 1 |
|---|---|---|
| Sheet name | Ink used | second |
| Disinfectant visualizing sheet 1 | Example 1 | A |
| Disinfectant visualizing sheet 2 | Example 2 | A |
| Disinfectant visualizing sheet 3 | Example 3 | A |
| Disinfectant visualizing sheet 4 | Example 4 | A |
| Disinfectant visualizing sheet 5 | Example 5 | A |
| Disinfectant visualizing sheet 6 | Example 6 | A |
| Disinfectant visualizing sheet 7 | Example 7 | A |
| Disinfectant visualizing sheet 8 | Example 8 | A |
| Disinfectant visualizing sheet 9 | Example 9 | B |
| Disinfectant visualizing sheet 10 | Comparative | C |
| | Example 1 | |

A: 30 or more
B: 15 or more and less than 30
C: Less than 15

The ink according to the exemplary embodiment of the present invention makes it possible to visually confirm that the disinfectant has been applied.

### Evaluation 2 of Examples: Inkjet Discharge by Thermal Energy

The ink of Example 1 was charged into an ink cartridge and set in an inkjet recording apparatus (PIXUS iP7230 manufactured by Canon Inc.) that discharges ink from a recording head by the action of thermal energy. In this example, a solid image recorded by applying eight 2.5-pL ink droplets to a unit area of 1/600 inch× 1/600 inch is defined as having a “recording duty of 100%”. A solid image with a recording duty of 100% was recorded using this inkjet recording apparatus in an environment with a temperature of 23° C. and a relative humidity of 55%.

The present invention is not limited to the above-described exemplary embodiments, and various modifications and variations can be made without departing from the spirit and scope of the present invention. Therefore, the following claims are appended to apprise the public of the scope of the present invention.

The above disclosure includes the following configurations.

### (Configuration 1)

A disinfectant visualizing ink having a visualizing agent that changes in color state when a disinfectant is applied and is hardly soluble in an aqueous liquid, a dispersant that disperses the visualizing agent in the aqueous liquid, and the aqueous liquid.

### (Configuration 2)

The disinfectant visualizing ink according to configuration 1, wherein the dispersant has at least one functional group selected from the group consisting of a polyethylene oxide group, a polypropylene oxide group, and a polyglycerin group.

### (Configuration 3)

The disinfectant visualizing ink according to configuration 1 or 2, wherein the dispersant has a copolymer containing a repeating unit derived from ethylene oxide and a repeating unit derived from propylene oxide.

### (Configuration 4)

The disinfectant visualizing ink according to any one of configurations 1 to 3, wherein when a total mass of the visualizing agent contained in the ink is taken as 100% by mass, a content of the dispersant in the ink is 30% by mass or more and 200% by mass or less.

### (Configuration 5)

The disinfectant visualizing ink according to any one of configurations 1 to 4, wherein the visualizing agent contains at least one selected from the group consisting of orthocresolphthalein, thymol blue, and thymolphthalein.

### (Configuration 6)

The disinfectant visualizing ink according to configuration 1, wherein when a total mass of the ink is taken as 100% by mass, a content of the visualizing agent in the ink is 1% by mass or more and 10% by mass or less.

### (Configuration 7)

The disinfectant visualizing ink according to configuration 1, wherein the aqueous liquid is water or a mixed solvent of water and a water-soluble organic solvent.

### (Configuration 8)

A disinfectant visualizing sheet including a base material and a chromogenic portion provided on the base material, wherein the chromogenic portion 101 has a visualizing agent according to any one of configurations 1 to 7.

### (Configuration 9)

The disinfectant visualizing sheet according to any one of configurations 1 to 8, wherein the chromogenic portion has a holding portion that holds the disinfectant.

### (Configuration 10)

The disinfectant visualizing sheet according to configuration 9, wherein the holding portion contains a plurality of inorganic particles, and a void is formed between the plurality of inorganic particles.

### (Configuration 11)

The disinfectant visualizing sheet according to configuration 9 or 10, wherein the holding portion contains at least one selected from the group consisting of pearl necklace-shaped silica particles, spherical colloidal silica, non-spherical colloidal silica, alumina particles, titania particles, and zirconia particles.

### (Configuration 12)

The disinfectant visualizing sheet according to any one of configurations 8 to 11, wherein the chromogenic portion has a binder.

### (Configuration 13)

The disinfectant visualizing sheet according to any one of configurations 8 to 12, wherein a protection portion is provided on the chromogenic portion.

The present invention is not limited to the above-described exemplary embodiments, and various modifications and variations can be made without departing from the spirit and scope of the present invention. Therefore, the following claims are appended to apprise the public of the scope of the present invention.

According to a disinfectant visualizing ink of the present invention, it is possible to provide ink that allows visual confirmation that a disinfectant has been applied.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A disinfectant visualizing ink comprising:
a visualizing agent that changes in color state when a disinfectant is applied and is hardly soluble in an aqueous liquid;
a dispersant that disperses the visualizing agent in the aqueous liquid; and
the aqueous liquid.

2. The disinfectant visualizing ink according to claim 1, wherein the dispersant has at least one functional group selected from the group consisting of a polyethylene oxide group, a polypropylene oxide group, and a polyglycerin group.

3. The disinfectant visualizing ink according to claim 1, wherein the dispersant has a copolymer containing a repeating unit derived from ethylene oxide and a repeating unit derived from propylene oxide.

4. The disinfectant visualizing ink according to claim 1, wherein when a total mass of the visualizing agent contained in the ink is taken as 100% by mass, a content of the dispersant in the ink is 30% by mass or more and 200% by mass or less.

5. The disinfectant visualizing ink according to claim 1, wherein the visualizing agent contains at least one selected from the group consisting of orthocresolphthalein, thymol blue, and thymolphthalein.

6. The disinfectant visualizing ink according to claim 1, wherein when a total mass of the ink is taken as 100% by mass, a content of the visualizing agent in the ink is 1% by mass or more and 10% by mass or less.

7. The disinfectant visualizing ink according to claim 1, wherein the aqueous liquid is water or a mixed solvent of water and a water-soluble organic solvent.

8. A disinfectant visualizing sheet comprising:
a base material; and
a chromogenic portion provided on the base material,
wherein the chromogenic portion has the visualizing agent according to claim 1.

9. The disinfectant visualizing sheet according to claim 8, wherein the chromogenic portion has a holding portion that holds the disinfectant.

10. The disinfectant visualizing sheet according to claim 9, wherein the holding portion contains a plurality of inorganic particles, and a void is formed between the plurality of inorganic particles.

11. The disinfectant visualizing sheet according to claim 9, wherein the holding portion contains at least one selected from the group consisting of pearl necklace-shaped silica particles, spherical colloidal silica, non-spherical colloidal silica, alumina particles, titania particles, and zirconia particles.

12. The disinfectant visualizing sheet according to claim 8, wherein the chromogenic portion has a binder.

13. The disinfectant visualizing sheet according to claim 8, wherein a protection portion is provided on the chromogenic portion.
